(19) United States
(12) Patent Application Publication
(10) Pub. No.: US 2020/0368141 A1
(43) Pub. Date: Nov. 26, 2020

(54) Ultraviolet Ray Blocking Mixture

(71) Applicant: Edward Jonas Domanskis, Los Angeles, CA (US)

(72) Inventor: Edward Jonas Domanskis, Los Angeles, CA (US)

(21) Appl. No.: 16/418,677

(22) Filed: May 21, 2019

(51) Int. Cl. A61K 8/9789; A61Q 17/04; A61K 8/06

(52) U.S. Cl. CPC A61K 8/9789; A61Q 17/04; A61K 8/19; A61K 2800/412; A61K 2800/805; A61K 8/062

(57) ABSTRACT

A sunscreen made by combining organic or inorganic matter, such as rose petals, leaves from various plants, cardboard, or combinations thereof, with a carrier, such as a lotion or cream base, that has no physical blocking component of ultraviolet rays on its own. The combination or mixture results in no chemical reaction of the components and can be separated back into its original components. The leaves or other products of plants, by coalescing or overlapping in a carrier, physically block the rays of the sun with a higher concentration of these elements causing a further coalescence of the pieces providing a higher SPF and greater sun protection. These particles of plants or other materials simply block the rays much like a window shade blocks sunlight.

### FIELD OF THE INVENTION

The present invention pertains generally to ultraviolet-ray-blocking substances for use in skin protection. More particularly, the present invention pertains to an ultraviolet ray blocking mixture. The Present invention is particularly, but not exclusively, useful as a sunscreen.

### BACKGROUND OF THE INVENTION

Sunscreens are commonly used to protect skin from harmful ultraviolet (UV) radiation. This includes Ultraviolet A radiation (UVA) with a wavelength from 315 to 400 nanometers, which can penetrate the skin and cause premature aging, and Ultraviolet B radiation (UVB) with a wavelength from 280 to 315 nanometers, which does not penetrate deeply into the skin, but causes sunburn on the surface of the skin. Ultraviolet C radiation, with shorter wavelengths of 280 nm or less, is absorbed by the atmosphere.

Sun Protection Factor (SPF) is a measurement of the proportion of ultraviolet rays that are prevented from reaching the skin. More particularly, for a given number n, when using a sunscreen of SPF n, only 1/n of the radiation that would reach the skin without sunscreen actually reaches the skin.

Currently available sunscreens have a zinc oxide, titanium dioxide, or benzones, or a combination thereof as active ingredients. These active ingredients work by either absorbing the UVA and UVB rays, as does zinc, or reflecting them, as does titanium. They may be combined in different proportions to increase the SPF rating. These active ingredients are then combined with different inactive ingredients, but the latter cause no increase of sun protection. Some of the chemicals used are carcinogenic and environmentally unfriendly.

Zinc oxide and titanium dioxide appear white on the skin. In order to avoid this appearance, manufacturers use zinc oxide and titanium dioxide in the form of nanoparticles usually sized in the tens of nanometers in diameter (e.g., from around 10 to 60 nanometers in the case of zinc oxide, or from around 5 to 50 nanometers or titanium dioxide) before being combined or coated with other compounds. Research has shown that at these sizes, Zinc oxide and titanium dioxide each can damage cellular DNA through the generation of free radicals when exposed to light. Such damage has been shown in isolated cell experiments, and it is unclear whether it presents a risk to sunscreen users. It is widely believed that a risk would only be present if the zinc oxide or titanium dioxide nanoparticles were to penetrate into the skin beyond the stratum corneum, or outer layer of corneocytes (the dead cells forming the outer skin). Although most studies have suggested that zinc oxide and titanium dioxide nanoparticles do not penetrate the skin, a small number of studies have found that at least small amounts of zinc oxide or titanium dioxide penetrate the outer layers of the skin. Moreover, most of the existing studies dealt with healthy skin, and thus leave uncertain whether ordinary cuts, scrapes, or even minor sunburn can increase the amount of skin absorption.

Zinc oxide, titanium dioxide, and benzones used in sunscreen also tends to enter wastewater when the sunscreen is washed off in showers or swimming pools, and they can enter directly into the environment when a sunscreen user swims in a beach, river, or lake. Studies have implicated benzones from sunscreen in coral reef bleaching, and oxybenzone is known to be toxic to other sea life. Zinc oxide and titanium dioxide may be less toxic to marine life in particle sizes greater than nanoparticles, but in nanoparticle sizes they appear to exhibit toxicity to marine life including causing coral reef bleaching. Titanium dioxide is also known to be toxic to soil invertebrates. The full extent of the environmental impact of commercially available sunscreens may as yet be unknown, and ongoing research in the area is needed. Nonetheless, a negative impact is sufficiently well known that the State of Hawaii enacted legislation prohibiting the sale of sunscreens containing oxybenzone after Jan. 21, 2021, without a prescription from a healthcare provider. Similar prohibitions on sunscreens containing oxybenzone have been enacted in the city of Key West, Fla., and in Palau.

Safety is also an important issue in preparing sunscreen, as well as cosmetics in general. It is not generally predictable prior to testing what harms might result from cosmetic components, either to the user or to the environment. The U.S. Food and Drug Administration (FDA) has received thousands of complaints, including hair loss, itching, rashes, allergic reactions, and even cancer, resulting from the use of cosmetics that were presumed safe at the time they were originally offered to the public. These adverse events are underreported since cosmetics manufacturers are not required to forward complaints they receive to the FDA. For example, the FDA received 1,386 reports of adverse reactions, primarily hair loss, from customers who used WEN by Chaz Dean Cleansing Conditioners up to Nov. 15, 2016; during investigation, it was discovered that the companies marketing the products had received more than 21,000 complaints.

In light of the above, it would be advantageous to provide a sunscreen using alternative substances to zinc oxide, titanium dioxide, and benzones to block the rays of the sun. It would be further advantageous to provide a sunscreen having environmentally friendly and human-safe components, and more particularly a sunscreen that is nontoxic not only to the user, but also to marine life and soil vertebrates. It would be further advantageous to provide an environmentally friendly sunscreen having a neutral color.

### SUMMARY OF THE INVENTION

Disclosed are sunscreens made by the combination of organic or inorganic matter, such as rose petals, leaves from various plants, cardboard, or combinations thereof, with a carrier, such as a lotion or cream base, that has no physical blocking component of ultraviolet rays on its own. The combination or mixture results in no chemical reaction of the components and can be separated back into its original components.

The leaves or other products of plants, by coalescing or overlapping in a carrier, physically block the rays of the sun with a higher concentration of these elements causing a further coalescence of the pieces providing a higher SPF and greater sun protection. These particles of plants or other materials simply block the rays much like a window shade blocks sunlight. The greater the size of the particles used in the mixture, the greater the surface area it would cover and the greater expected sun protection. The limitation is the materials immersibility into the carrier (e.g., lotion) and its cosmetic limitation: color on skin, as well as how it would feel. A preferred embodiment uses rose petals, which are smoother on the skin than leaves or cardboard, the latter being less viscous in a mixture and more apparent to sight and feel. Additional embodiments use multiple materials to block the rays.

The choice of UV-blocking matter is critical, since even organic plant matter from certain plants may have environmental and health-related impacts. Many plants contain skin irritants, while others, such as eucalyptus, may provide significant health benefits when applied topically. While some harmful plants are well-known—certainly no serious cosmetic provider would consider poison oak as a UV-blocking component of a cream or lotion—it is not generally predictable prior to testing what harms might result from cosmetic components, either to the user or to the environment, as discussed above. When using plant matter or other UV-blocking matter for a UV ray blocking mixture, it is important to select parts of a plant that is neutral or beneficial in its effects when applied to human skin, and at the same time is ecologically safe when it enters wastewater, or directly into oceans, rivers, or lakes.

Plants and plant parts safe for use on the skin and generally safe for the environment include Camellia sinensis (tea), Cvena sativa (oats), Bambusa vulgaris (bamboo) leaves and stems, and seaweed—especially kelp, fucus vesiculosus (bladderwrack), and other edible varieties, and excepting the dermatitis-causing lyngbya majuscule (which is another example of the unpredictability of safety issues, since other species of seaweed are generally harmless), as well as rose petals, and juglans regia (walnut) shells. In some cases, benefits such as clearer, healthier skin have been reported.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 illustrates an exemplary process for preparing an ultraviolet ray blocking mixture of the present invention;

FIG. 2 illustrates an alternate exemplary process for preparing an ultraviolet ray blocking mixture of the present invention; and

FIG. 3 illustrates an ultraviolate ray blocking mixture applied to skin and blocking ultraviolet rays from contacting and damaging the skin.

### DETAILED DESCRIPTION

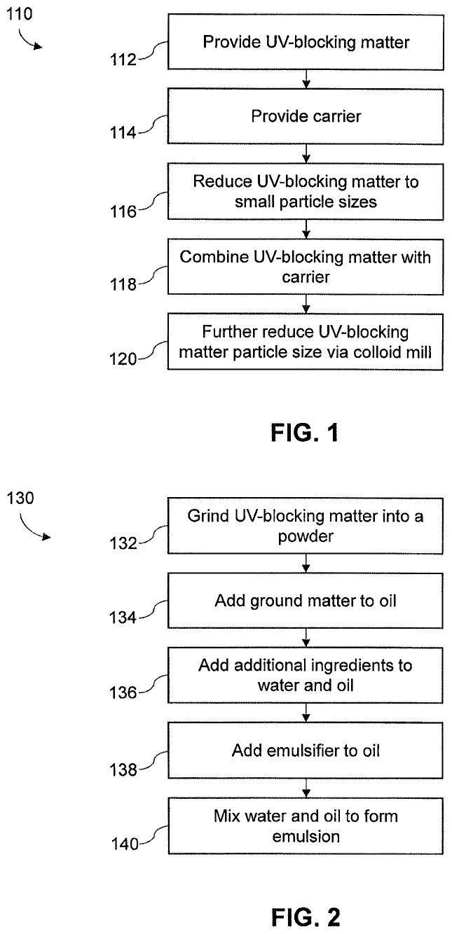

Referring initially to FIG. 1, a preferred embodiment of a process for preparing an ultraviolet ray blocking mixture is illustrated and generally labeled 110. Process 110 includes step 112 of providing UV-blocking matter, which is organic or inorganic solid matter. In a preferred embodiment the UV-blocking matter is plant matter, such as leaves or flower petals of selected plants, or a combination of matter from various plants and/or other organic and inorganic matter. In step 114, a carrier is provided, such as a cream or a lotion.

In step 116 the UV-blocking matter is reduced to small particle sizes. In particular, the size of the particles of the UV-blocking matter should be sufficiently small to allow for a substantially uniform distribution of the organic matter throughout the carrier. Preferably, the diameter of the particles would be on the order of one millimeter or less. The reduction of the UV-blocking matter to small particle sizes is performed by milling, crushing, cutting, or any other method or combination of methods known in the art.

In step 118, the UV-blocking matter is combined with the carrier. The UV-blocking matter and carrier are mixed together to result in a substantially uniform distribution of the UV-blocking matter throughout the carrier. By a “substantially uniform” distribution, it is meant that the particles of organic matter are distributed in a sufficiently uniform matter that the smallest amount of the mixture a consumer is likely to apply will contain particles of organic matter in a proportion to the applied mixture that is roughly equivalent to the proportion of particles of organic matter to mixture in the entire volume of the product.

In some, but not all, preferred embodiments, step 120 is performed, in which the particle size of the organic matter in the mixture is further reduced by processing the mixture in a colloid mill or related equipment for reducing particle sizes of mixtures known in the art. However, the particle size is critical, since the UV-ray blocking properties may be reduced if the particle size is too small. Thus, preferred embodiments use particle sizes of greater than one hundred (100) nanometers in diameter. Among exemplary embodiments, one embodiment includes particle sizes of about one (1) to ten (10) micrometers, another embodiment includes particle sizes of about ten (10) to fifty (50) micrometers, another embodiment includes particle sizes of about fifty (50) to one-hundred-fifty (150) micrometers, and another includes particle sizes above one-hundred-fifty (150) micrometers but less than one millimeter. Fifty (50) to five-hundred (500) micrometers is a preferred range of particle sizes. An alternative embodiment uses particle sizes of approximately ten (10) micrometers, about the size of a water droplet in a cloud, which is sufficient to block the passage of light. More generally, the larger the particle size, the better the UV-blocking effect. Thus, the ideal particle size is the largest size that feels smooth on the skin and does not affect skin color. This increases the possibility of overlap of the particulate matter, and so increases the protection from UV rays while decreasing the penetration of both UV rays and the particulate matter into the skin. These size considerations related to the size of pieces of “particles” of matter used for UV-ray blocking in the mixture apply across the various embodiments of the ultraviolet ray blocking mixture described herein.

Referring now to FIG. 2, an alternate preferred embodiment of a process for preparing an ultraviolet ray blocking mixture is illustrated and generally labeled 130. In this embodiment, the UV-blocking matter is added during the preparation of the carrier, rather than to an existing carrier. In preferred embodiments, the carrier is a lotion or cream.

In step 132 UV-blocking matter which may be organic or inorganic solid matter, is ground into a powder. In a non-limiting preferred embodiment, the UV-blocking matter is plant matter, such as leaves or flower petals of selected plants, or a combination of matter from various plants. The ground matter is added to an oil component of the carrier in step 134. Non-limiting preferred embodiments of the oil component include one or more natural plant oils, such as carthamus tinctorius (safflower) seed oil, prunus amygdalus dulcis (sweet almond) oil, and hypericum perforatum (St. John's wort) oil.

Additional ingredients are also added to the oil, to a water component of the carrier, or both in step 136, which may be performed before, after, or contemporaneously with step 134. Each ingredient is added either to the water component or the oil component based on whether it is better dispersed in water or oil. For example, some preferred embodiments of the carrier include shea butter, cocoa butter, or both, which are added to the oil component. Xanthan gum is also added in a preferred embodiment both as a thickener and a stabilizer.

In step 138, one or more emulsifiers are added to the oil component, the water component, or both. In a preferred embodiment, this step includes the addition of emulsifying wax to the oil component.

In step 140, the oil and water components are mixed to form an emulsion. The end result is a lotion or cream which acts as a carrier for the UV-blocking matter, its thickness depending on the proportion of water to oil, the thickening agents, and other ingredients.

Any of the processes used in some cases involve an additional step of boiling either the particulate matter before addition to the other ingredients, or boiling the mixture as a whole. This allows the matter to be broken down to be usable with the carrier, and in some cases may make the matter more suitable for topical use by breaking down skin irritants or other potentially adverse components. Such is the case, for example, with at least one species of bamboo.

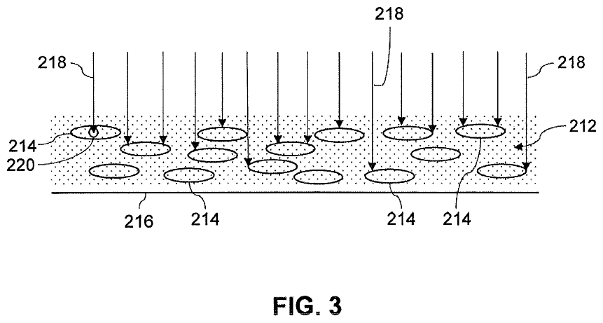

Referring now to FIG. 3, a carrier 212, such as a lotion or a body cream, containing particles 214 of UV-blocking matter, as applied to skin 216 is illustrated. Particles 214 dispersed throughout the carrier 212 block the passage of ultraviolet rays 218, preventing them from reaching the skin. A non-limiting preferred embodiment uses rose petals for at least a portion of particles 214.

In embodiments in which particles 214 include plant matter, and especially leaves, the plant-based particles 214 may contain some amount of light-absorbing pigment 220 (for example, chlorophyll). These pigments 220 in some cases may absorb a certain amount of ultraviolet radiation, thus providing an alternative mechanism for protection from UV rays 218. However, ultraviolet light is known to bleach or otherwise damage chlorophyll and related compounds; this results in a previously unrecognized problem that any UV protection through absorption by pigments 220 is limited in duration. Plant-based sunscreen formulas currently available or otherwise disclosed publicly suffer from this limitation, since their plant content is based on oils or matter that is too finely ground to provide optimum blocking of UV rays 218. As a result, when the chlorophyll in currently available plant-based sunscreens becomes damaged by UV light, the effectiveness of those sunscreens is significantly lowered; in other words, their SPF is significantly lowered after a short period of use. The larger particle sizes disclosed herein and used in preferred embodiments of the present invention avoid this problem.

While there have been shown what are presently considered to be preferred embodiments of the present invention, it will be apparent to those skilled in the art that various changes and modifications can be made herein without departing from the scope and spirit of the invention.

1. A UV-blocking mixture, comprising:
a plurality of UV-blocking particles; and
a carrier,
wherein the plurality of UV-blocking particles is uniformly distributed throughout the carrier.

2. The UV-blocking mixture as recited in claim 1, wherein the carrier comprises a lotion or a cream.

3. The UV-blocking mixture as recited in claim 2, wherein each particle of the plurality of UV-blocking particles comprises a diameter between fifty and five-hundred micrometers in size.

4. The UV-blocking mixture as recited in claim 3, wherein each particle of the plurality of UV-blocking particles comprises a diameter of at least one-hundred fifty micrometers in size.

5. The UV-blocking mixture as recited in claim 3, wherein the plurality of UV-blocking particles comprises particles of plant matter.

6. The UV-blocking mixture as recited in claim 5, wherein the particles of plant matter comprise leaves.

7. The UV-blocking mixture as recited in claim 6, wherein the particles of plant matter comprise tea leaves.

8. The UV-blocking mixture as recited in claim 5, wherein the particles of plant matter comprise flower petals.

9. The UV-blocking mixture as recited in claim 8, wherein the particles of plant matter comprise rose petals.

10. The UV-blocking mixture as recited in claim 3, wherein the UV-blocking particles comprise inorganic matter.

11. The UV-blocking mixture as recited in claim 3, wherein the UV-blocking particles comprise a combination or organic and inorganic matter.

12. A method of preparing a UV-blocking mixture, comprising the steps of:
providing UV-blocking matter;
providing a carrier;
reducing the UV-blocking matter to sufficiently small particle sizes to allow for a substantially uniform distribution of the UV-blocking matter throughout the carrier; and
combining the UV-blocking matter with the carrier.

13. The method of preparing a UV-blocking mixture as recited in claim 12, further comprising the step of:
using a colloid mill to further reduce the size of the UV-blocking matter.

14. The method of preparing a UV-blocking mixture as recited in claim 12, wherein the step of reducing the UV-blocking matter results in a particle size of between fifty and five-hundred micrometers in diameter.

15. The method of preparing a UV-blocking mixture as recited in claim 14, wherein the step of reducing the UV-blocking matter results in a particle size of at least one-hundred fifty micrometers in diameter.

16. The method of preparing a UV-blocking mixture as recited in claim 12, wherein the UV-blocking matter comprises organic plant matter.

17. A method of preparing a UV-blocking mixture, comprising the steps of:
providing water;
providing oil;
grinding UV-blocking matter into a powder;
adding the powder to the oil;
adding additional ingredients to the water and oil;
adding an emulsifier to the oil; and
mixing the water and oil to form an emulsion.

18. The method of preparing a UV-blocking mixture as recited in claim 17, wherein the step of grinding UV-blocking matter into a powder results in a powder having a particle size of between fifty and five-hundred micrometers in diameter.

19. The method of preparing a UV-blocking mixture as recited in claim 18, wherein the step of grinding UV-blocking matter into a powder results in a powder having a particle size of at least one-hundred fifty micrometers in diameter.

20. The method of preparing a UV-blocking mixture as recited in claim 17, wherein the UV-blocking matter comprises organic plant matter.
